(19) United States
(12) Patent Application Publication
(10) Pub. No.: US 2025/0157601 A1
(43) Pub. Date: May 15, 2025

(54) MEDICAL RECORD GENERATION FOR VIRTUAL NURSING

(71) Applicant: Welch Allyn, Inc., Skaneateles Falls, NY (US)

(72) Inventors: Gene J. Wolfe, Pittsford, NY (US); John A. Lane, Venice, FL (US); WonKyung McSweeney, Manlius, NY (US); David E. Quinn, Moravia, NY (US); Tyson B. Whitaker, Arden, NC (US)

(21) Appl. No.: 18/931,561

(22) Filed: Oct. 30, 2024

Related U.S. Application Data
(60) Provisional application No. 63/597,818, filed on Nov. 10, 2023.
(60) Provisional application No. 63/604,530, filed on Nov. 30, 2023.

(51) Int. Cl. G16H 10/60; G06T 11/00; G06V 10/764; G06V 20/40; G11B 27/34; G16H 80/00

(52) U.S. Cl. CPC G16H 10/60; G06T 11/00; G06V 10/764; G06V 20/44; G06V 20/49; G16H 80/00; G06T 2210/41; G06V 2201/10; G11B 27/34

(57) ABSTRACT

A system for providing a clinical analysis of a subject is described. The system receives video data and audio data of the subject. The system extracts metadata from the video data and the audio data. The metadata includes an event, one or more parameters measured during the event, and a time stamp of when the event occurs in the video data and the audio data. The system generates an electronic medical record that includes a video display area for providing playback of the video data and the audio data, and a data stream area that displays one or more medically relevant events in the video data and the audio data. Selection of a medically relevant event causes the video display area to skip to a portion of the video data and the audio data where the medically relevant event occurs based on the time stamp in the metadata.

### CROSS REFERENCE TO RELATED APPLICATIONS

This application claims priority to U.S. Provisional Patent Application No. 63/597,818, filed Nov. 10, 2023, and U.S. Provisional Patent Application No. 63/604,530, filed Nov. 30, 2023, the disclosures of which are hereby incorporated by reference in their entireties.

### BACKGROUND

An electronic medical record (EMR), sometimes alternatively called an electronic health record (EHR), stores patient health information in a digital format. EMRs can be shared across different healthcare facilities and settings. EMRs may include a range of patient health information including demographics, medical history, medication and allergies, immunization status, laboratory test results, radiology images, vital signs, personal statistics such as age and weight, and billing information. EMRs are not designed to facilitate a personalized human narrative, which can prevent an effective clinical analysis by a trained medical professional.

### SUMMARY

In general terms, the present disclosure relates to electronic medical records. In one possible configuration, an electronic medical record is generated to include a video display area and identification of medically relevant events for clinical analysis. Various aspects are described in this disclosure, which include, but are not limited to, the following aspects.

One aspect relates to a system for generating an electronic medical record, the system comprising: at least one processing device; and at least one computer readable data storage device storing software instructions that, when executed by the at least one processing device, cause the system to: receive video data and audio data of the subject; extract metadata from the video data and the audio data, the metadata including an event, one or more parameters measured during the event, and a time stamp of when the event occurs in the video data and the audio data; and generate an electronic medical record that includes a video display area for providing playback of the video data and the audio data, and a data stream area that displays one or more events in the video data and the audio data, at least one event of the one or more events when selected causes the video display area to skip to a portion of the video data and the audio data where the at least one event occurs based on the time stamp in the metadata associated with the at least one event, and wherein the at least one event is identified based on the metadata as relevant to the clinical analysis of the subject.

Another aspect relates to a method of generating an electronic medical record, the method comprising: receiving video data and audio data of the subject; extracting metadata from the video data and the audio data, the metadata including an event, one or more parameters measured during the event, and a time stamp of when the event occurs in the video data and the audio data; and generating the electronic medical record that includes a video display area for providing playback of the video data and the audio data, and a data stream area that displays one or more events in the video data and the audio data, at least one event of the one or more events when selected causes the video display area to skip to a portion of the video data and the audio data where the at least one event occurs based on the time stamp in the metadata.

Another aspect relates to a device for providing a clinical analysis of a subject, the device comprising: at least one processing device; and at least one computer readable data storage device storing software instructions that, when executed by the at least one processing device, cause the at least one processing device to: conduct a data acquisition session including presenting interrogatories to guide acquisition of video data and audio data from the subject; capture the video data and the audio data from the subject; determine one or more vital signs and physical assessment attributes of the subject based on the video data and the audio data captured from the subject; and extract metadata from the video data and the audio data, the metadata including an event, one or more parameters measured during the event, and a time stamp of when the event occurs in the video data and the audio data.

Another aspect relates to a system for generating an electronic medical record, the system comprising: at least one processing device; and at least one computer readable data storage device storing software instructions that, when executed by the at least one processing device, cause the system to: receive a health condition of a subject; receive raw data including at least video data captured of the subject; extract landmarks from an anatomy shown in the video data based on the health condition of the subject; generate synthetic data including at least a synthetic anatomy constructed based on the landmarks extracted from the anatomy shown in the video data; and store the landmarks in the electronic medical record of the subject.

Another aspect relates to a method of generating an electronic medical record, the method comprising: receiving a health condition of a subject; receiving raw data including at least video data captured of the subject; extracting landmarks from an anatomy shown in the video data based on the health condition of the subject; generating synthetic data including at least a synthetic anatomy constructed based on the landmarks extracted from the anatomy shown in the video data; and storing the landmarks in the electronic medical record of the subject.

Another aspect relates to a device for capturing medically relevant data from a subject, the device comprising: at least one processing device; and at least one computer readable data storage device storing software instructions that, when executed by the at least one processing device, cause the device to: conduct a data acquisition session with the subject, the data acquisition session including interrogatories to guide capture of video data and audio data based on a health condition of the subject; extract landmarks from an anatomy in the video data based on the health condition of the subject; extract sounds from the audio data based on the health condition of the subject; and generate synthetic data including at least a synthetic anatomy constructed based on the landmarks extracted from the anatomy shown in the video data and a synthetic voice based on the sounds extracted from the audio data.

A variety of additional aspects will be set forth in the description that follows. The aspects can relate to individual features and to combination of features. It is to be understood that both the foregoing general description and the following detailed description are exemplary and explanatory only and are not restrictive of the broad inventive concepts upon which the embodiments disclosed herein are based.

### DESCRIPTION OF THE FIGURES

The following drawing figures, which form a part of this application, are illustrative of the described technology and are not meant to limit the scope of the disclosure in any manner.

FIG. 1 illustrates an example of a system for generating an electronic medical record for providing a clinical analysis of a subject.

FIG. 2 schematically illustrates an example of a data acquisition device that is part of the system of FIG. 1.

FIG. 3 schematically illustrates an example of an analytics system that is part of the system of FIG. 1.

FIG. 4 illustrates an example of a graphical user interface displayed on the data acquisition device of FIG. 2.

FIG. 5 schematically illustrates an example of an electronic medical record (EMR) generated by the system of FIG. 1.

FIG. 6 illustrates an example of an EMR generated by the system of FIG. 1.

FIG. 7 schematically illustrates an example of a method of providing virtual nursing through generating an EMR that can be performed by the system of FIG. 1.

FIG. 8 schematically illustrates an example of a method of generating a video recording that can be performed by the system of FIG. 1.

FIG. 9 schematically illustrates an example of a method of performing a clinical assessment that can be performed by the system of FIG. 1.

FIG. 10 schematically illustrates another example of the system of FIG. 1 that can further enhance the EMR.

FIG. 11 schematically illustrates an example of medically relevant data extracted by a data extractor included in the example of the system in FIG. 10.

FIG. 12 schematically illustrates an example of synthetic data generated by a data synthesizer included in the example of the system in FIG. 10.

FIG. 13 schematically illustrates an example of synthetic data generated by the data synthesizer of FIG. 12.

FIG. 14 schematically illustrates another example of synthetic data generated by the data synthesizer of FIG. 12.

FIG. 15 schematically illustrates an example of a method of generating synthetic data for playback in the EMR that can be performed by the example of the system in FIG. 10.

### DETAILED DESCRIPTION

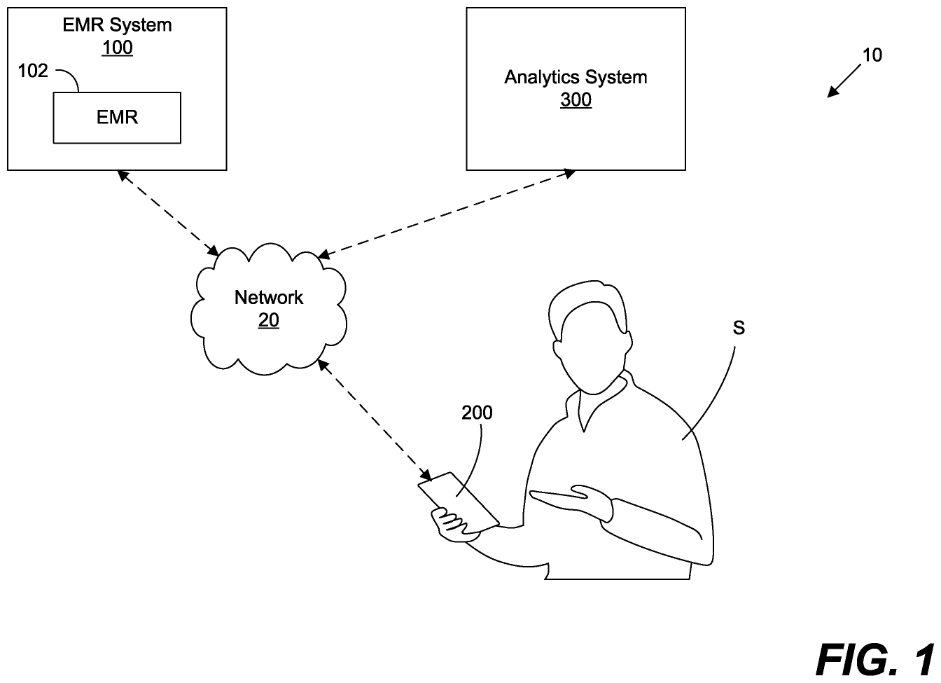

FIG. 1 illustrates an example of a system 10 for generating an electronic medical record (EMR) 102 for providing a clinical analysis of a subject S. The EMR 102 includes a configuration of health information that is not available in typical electronic medical records for providing a personalized human narrative to facilitate clinical assessment of the subject S.

The system 10 includes an EMR system 100 for storing the EMR 102 of the subject S. In addition to the EMR 102 of the subject S, the EMR system 100 stores a plurality of EMRs for a plurality of subjects. The EMRs can be shared across multiple healthcare facilities and settings over a communications network 20 for viewing by medical professionals. The communications network 20 can include any type of wired or wireless connections or any combinations thereof. The communications network 20 can include an intranet such as a computer network for sharing information, communication, collaboration tools, operational systems, and other computing services within an organization. Alternatively, the communications network 20 can include a public network, such as the Internet.

The system 10 includes a data acquisition device 200 that is operated by the subject S to capture image and audio data of the subject S. The data acquisition device 200 can be operated by the subject S in a medical facility such as a hospital, a long-term care facility, and the like when the subject S is a patient admitted to the medical facility. Also, the data acquisition device 200 can be operated by the subject S when in their home such that the subject S is not a patient admitted to a medical facility. As will be described in more detail below, the image and audio data captured by the data acquisition device 200 is used for generating the EMR 102 of the subject S to facilitate clinical assessment of the subject S when there are limited medical resources and/or when the subject S is located in a remote location such as their home. The EMR 102 includes a specialized configuration of heath information based on the image and audio data captured by the data acquisition device 200 when operated by the subject S. The data acquisition device 200 can communicate with the EMR system 100 over the communications network 20.

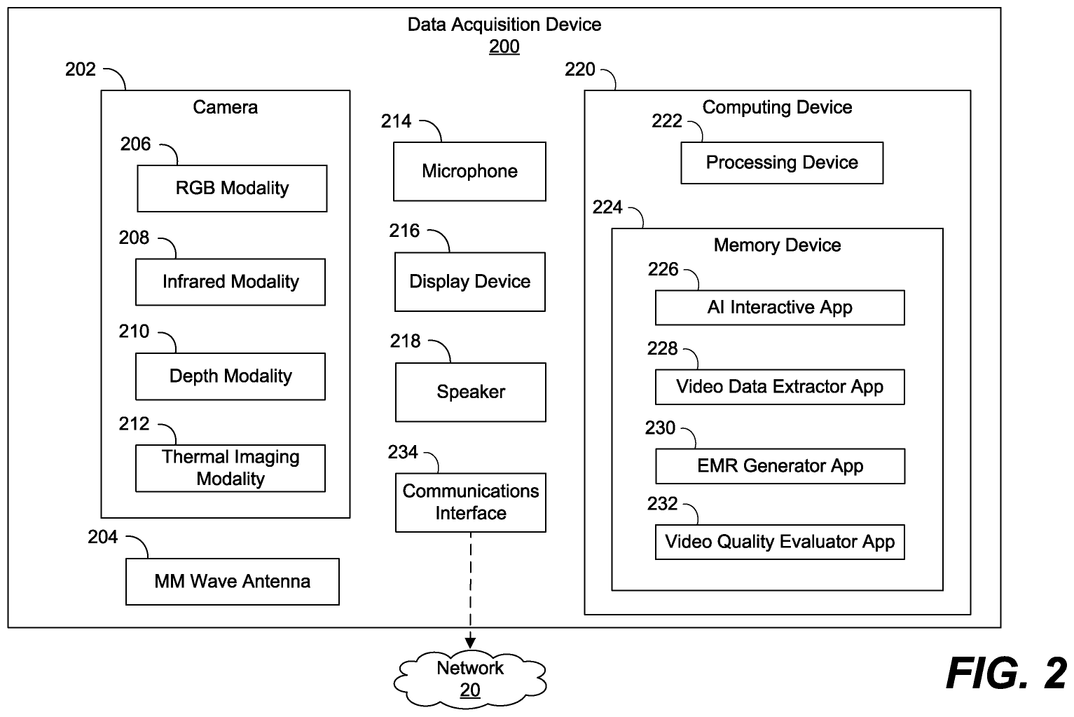

The data acquisition device 200 can include a portable computing device such as a tablet computer, a smartphone, or a laptop computer having an embedded camera and microphone for capturing the image and audio data of the subject S. Also, the data acquisition device 200 can include stationary computing devices such as a stationary monitor having an embedded camera and microphone for capturing the image and audio data of the subject S. Also, the data acquisition device 200 can include webcams, microphones, and other peripheral devices that connected to a separate computing device. The foregoing examples of the data acquisition device 200 are not exhaustive, and it is contemplated that the data acquisition device 200 can take any form or configuration that allows the capture of image and audio data of the subject S. The data acquisition device 200 is described in more detail with reference to FIG. 2.

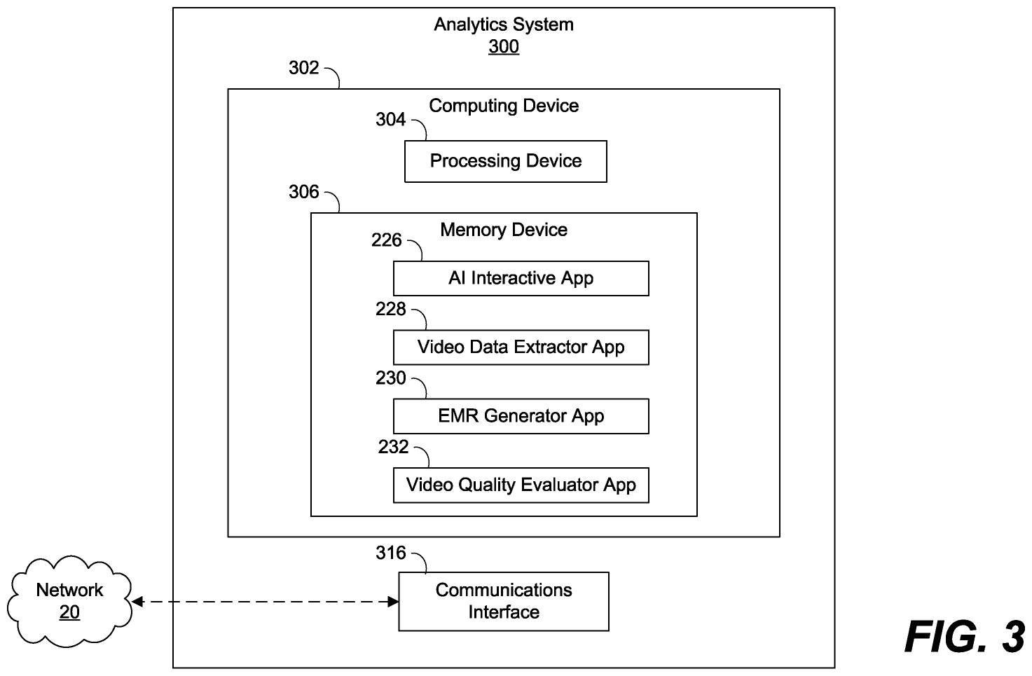

The system 10 further includes an analytics system 300 that can analyze the image and audio data captured by the data acquisition device 200. As will be described in more detail, the analytics system 300 can analyze the image and audio data captured by the data acquisition device 200 to guide a clinical assessment of the subject S. Additionally, the analytics system 300 can analyze the image and audio data captured by the data acquisition device 200 for generating the EMR 102 of the subject S. The analytics system 300 can communicate with the EMR system 100 and the data acquisition device 200 over the communications network 20. The analytics system 300 is described in more detail with reference to FIG. 3.

FIG. 2 schematically illustrates an example of the data acquisition device 200. As shown in FIG. 2, the data acquisition device 200 includes a camera 202 that includes one or more types of imaging modalities. For example, the camera 202 includes an RGB imaging modality 206 that can be used to capture a color video of the subject S. The camera 202 can also include an infrared imaging modality 208 that can be used to capture an infrared video of the subject S. The camera 202 can also include a depth imaging modality 210 that can be used to capture depth (D) data of the subject S as an output in real-time. The camera 202 can also include a thermal imaging modality 212 that can be used to capture a thermal imaging video of the subject S. The foregoing examples of the one or more imaging modalities of the camera 202 are not exhaustive, and it is contemplated that the camera 202 can include additional types of imaging modalities for capturing image and/or video data of the subject S. In some examples, two or more of the imaging modalities can be performed by the camera 202 simultaneously.

As further shown in FIG. 2, the data acquisition device 200 can also include a millimeter wave antenna 204 that can be used to determine a non-contact vital sign measurement based on the reflected millimeter wave signals of the subject S. The millimeter wave antenna 204 can include aspects of a patient monitoring device described in U.S. Pat. No. 11,653,848 B2, granted on May 23, 2023, which is incorporated herein by reference in its entirety. In some examples, one or more of the imaging modalities of the camera 202 and the millimeter wave measurements by the millimeter wave antenna 204 can be performed simultaneously.

The data acquisition device 200 includes a microphone 214 that can record audio of the subject S such as verbal responses to interrogatories. The microphone 214 can also record non-verbal sounds from the subject S including coughs, sneezes, groans, and the like.

The data acquisition device 200 can include a display device 216 and a speaker 218. As will be described in more detail, the display device 216 and the speaker 218 are controlled to present an avatar controlled by artificial intelligence for conducting a data acquisition session with the subject S. The data acquisition session can include a series of interrogatories to guide acquisition of video and audio data from the subject S. Thereafter, the video and audio data is used to generate the EMR 102. In some examples, the display device 216 can also be used to display the EMR 102 of the subject S. The display device 216 can include a touchscreen that operates to receive tactile inputs from the subject S such that the display device 216 is both a display device and a user input device. In some examples, the display device 216 is a liquid-crystal display (LCD), an organic light-emitting diode (OLED, a plasma panel, a quantum-dot light-emitting diode (QLED), or other type or combination of display screen technology.

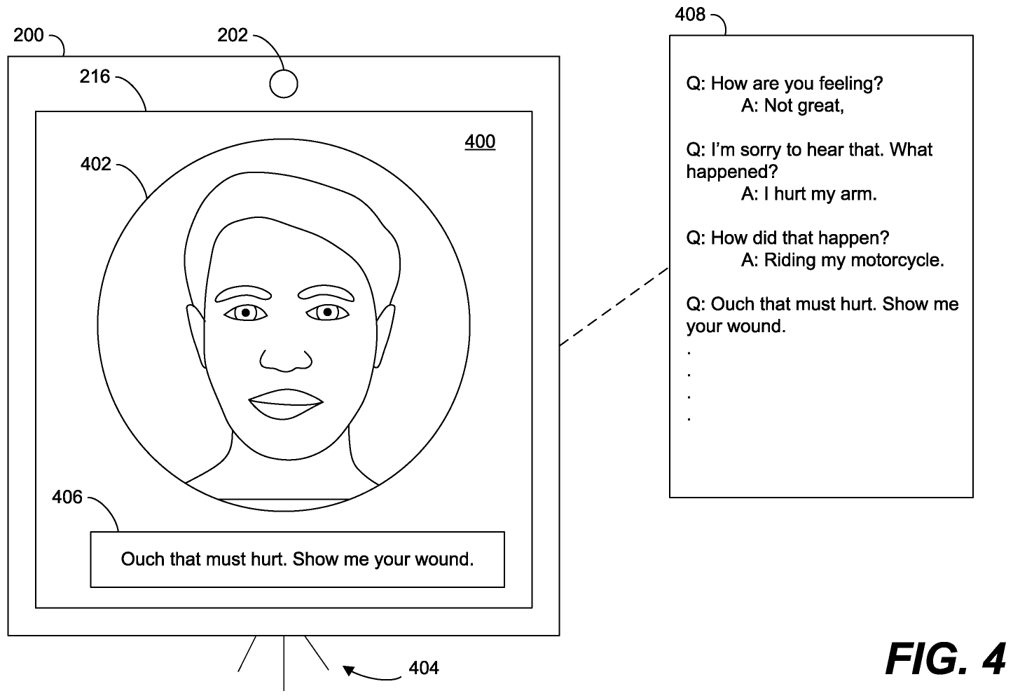

As an illustrative example of the data acquisition session performed by artificial intelligence, the display device 216 and the speaker 218 can be used to present interrogatories generated by artificial intelligence, and the microphone 214 can be used to record the verbal responses from the subject S to the interrogatories while the camera 202 records motor responses from the subject S to the interrogatories. For example, an avatar displayed on the display device 216 can ask the subject S to show an injury or wound, and the camera 202 can be used to capture a video of the subject S showing their injury or wound. An illustrative example of an interactive data acquisition session between the subject S and the avatar controlled by artificial intelligence is described in more detail with reference to FIG. 4.

As further shown in FIG. 2, the data acquisition device 200 includes a computing device 220 having at least one processing device 222 and a memory device 224. The at least one processing device 222 is an example of a processing unit such as a central processing unit (CPU). The at least one processing device 222 can include one or more central processing units (CPUs). In some examples, the at least one processing device 222 includes one or more digital signal processors, field-programmable gate arrays, and/or other types of electronic circuits.

The memory device 224 operates to store data and instructions for execution by the at least one processing device 222. In the example illustrated in FIG. 2, the memory device 224 stores one or more of an artificial intelligence (AI) interactive application 226, a video data extractor application 228, an electronic medical record (EMR) generator application 230, and a video quality evaluator application 232, each of which will be described in more detail. The memory device 224 includes computer-readable media, which may include any media that can be accessed by the data acquisition device 200. By way of example, computer-readable media include computer readable storage media and computer readable communication media.

Computer readable storage media includes volatile and nonvolatile, removable and non-removable media implemented in any device configured to store information such as computer readable instructions, data structures, program modules, or other data. Computer readable storage media can include, but is not limited to, random access memory, read only memory, electrically erasable programmable read only memory, flash memory, and other memory technology, including any medium that can be used to store information that can be accessed by the data acquisition device. The computer readable storage media is non-transitory.

Computer readable communication media embodies computer readable instructions, data structures, program modules or other data in a modulated data signal such as a carrier wave or other transport mechanism and includes any information delivery media. The term “modulated data signal” refers to a signal that has one or more of its characteristics set or changed in such a manner as to encode information in the signal. By way of example, computer readable communication media includes wired media such as a wired network or direct-wired connection, and wireless media such as acoustic, radio frequency, infrared, and other wireless media. Combinations of any of the above are within the scope of computer readable media.

The data acquisition device 200 further includes a communications interface 234 that allows the data acquisition device 200 to connect to the communications network 20. The communications interface 234 can include wired interfaces and wireless interfaces. For example, the communications interface 234 can wirelessly connect to the communications network 20 through cellular network communications, Wi-Fi, and other wireless connections. Alternatively, the communications interface 234 can connect to the communications network 20 using wired connections such as through an Ethernet or Universal Serial Bus (USB) cable.

FIG. 3 schematically illustrates an example of the analytics system 300. As shown in FIG. 3, the analytics system 300 includes a computing device 302 having at least one processing device 304 and a memory device 306. The description of the computing device 220 described above with reference to FIG. 2 can similarly apply to the computing device 302 of FIG. 3.

In the example illustrated in FIG. 3, the memory device 306 stores the AI interactive application 226, the video data extractor application 228, the EMR generator application 230, and the video quality evaluator application 232. In some examples, the analytics system 300 can perform one or more of the AI interactive application 226, the video data extractor application 228, the EMR generator application 230, and the video quality evaluator application 232, while the data acquisition device 200 performs one or more of the AI interactive application 226, the video data extractor application 228, the EMR generator application 230, and the video quality evaluator application 232. In other examples, the data acquisition device 200 captures raw image and audio data of the subject S, and the analytics system 300 performs the AI interactive application 226, the video data extractor application 228, the EMR generator application 230, and the video quality evaluator application 232. In further examples, the data acquisition device 200 captures image and audio data of the subject S, and the data acquisition device 200 performs the AI interactive application 226, the video data extractor application 228, the EMR generator application 230, and the video quality evaluator application 232. In such examples, the analytics system 300 is optional such that the system 10 may not include the analytics system 300.

The analytics system 300 further includes a communications interface 316 that allows the analytics system 300 to connect to the communications network 20. The communications interface 316 can include wired interfaces and wireless interfaces. For example, the communications interface 316 can wirelessly connect to the communications network 20 through cellular network communications, Wi-Fi, and other wireless connections. Alternatively, the communications interface 316 can connect to the communications network 20 using wired connections such as through an Ethernet or Universal Serial Bus (USB) cable.

FIG. 4 illustrates an example of a graphical user interface (GUI) 400 displayed on the display device 216 of the data acquisition device 200. The GUI 400 includes an avatar 402 that is controlled by the AI interactive application 226 to perform a data acquisition session with the subject S. An illustrative example of a data acquisition session is summarized in box 408. As shown in the example of FIG. 4, the AI interactive application 226 causes the speaker 218 of the data acquisition device 200 to output audio 404 that includes interrogatories asking the subject S about their health condition. While the subject S engages with the AI interactive application 226, the camera 202 and the microphone 214 capture image and audio data of the subject S stored as a video file. In some instances, the GUI 400 displays text 406 to provide closed captioning of the audio interrogatories that can be helpful for the subject S to engage with the AI interactive application 226 such as when the subject S has a hearing impairment.

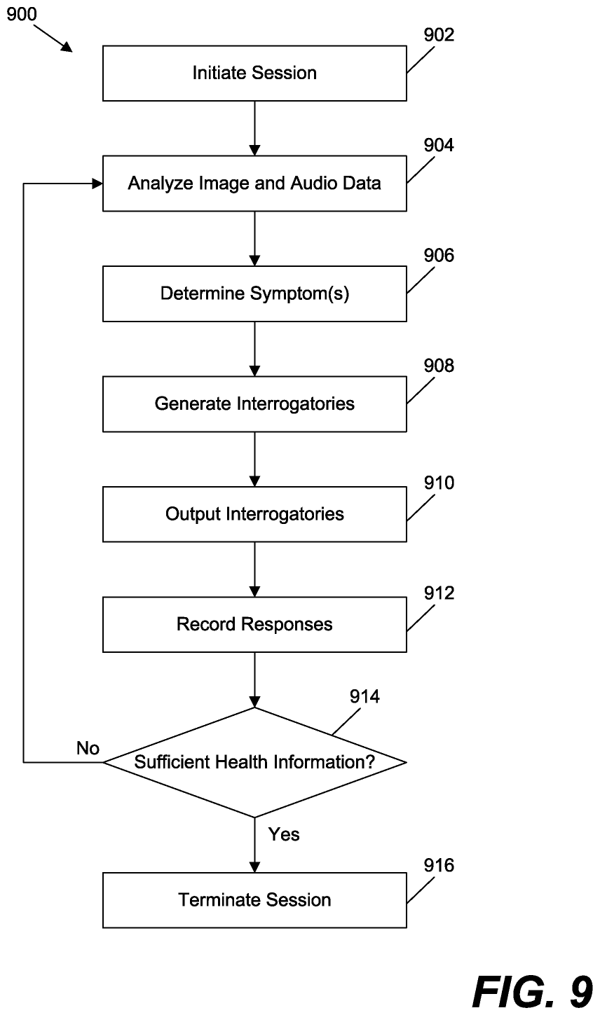

Based on the verbal and/or nonverbal responses to the interrogatories given by the subject S, the AI interactive application 226 adapts the interrogatories such that the data acquisition session is non-structured. Instead, the data acquisition session is designed as a fluid and free-flowing conversation with the subject S. As an illustrative example, the data acquisition session summarized in box 408 starts with a simple questions such as “How are you feeling?” and when the subject S replies “Not great.”, the AI interactive application 226 can follow up with “I'm sorry to hear that. What happened?” In this illustrative example, the subject S replies “I hurt my arm” and the AI interactive application 226 can follow up with “How did that happen?” The subject S then responds with “Riding my motorcycle,” and the AI interactive application 226 can follow up with “Ouch that must hurt. Show me your wound.” The data acquisition session continues until the AI interactive application 226 determines that it has enough data to produce an output such as the EMR 102, a recommendation for the subject S such as to prescribe one or more follow up exams, tests, or referrals, or an action such as to issue an alert to a medical professional. The data acquisition session performed by the AI interactive application 226 will be described in more detail with reference to the method 900 illustrated in FIG. 9.

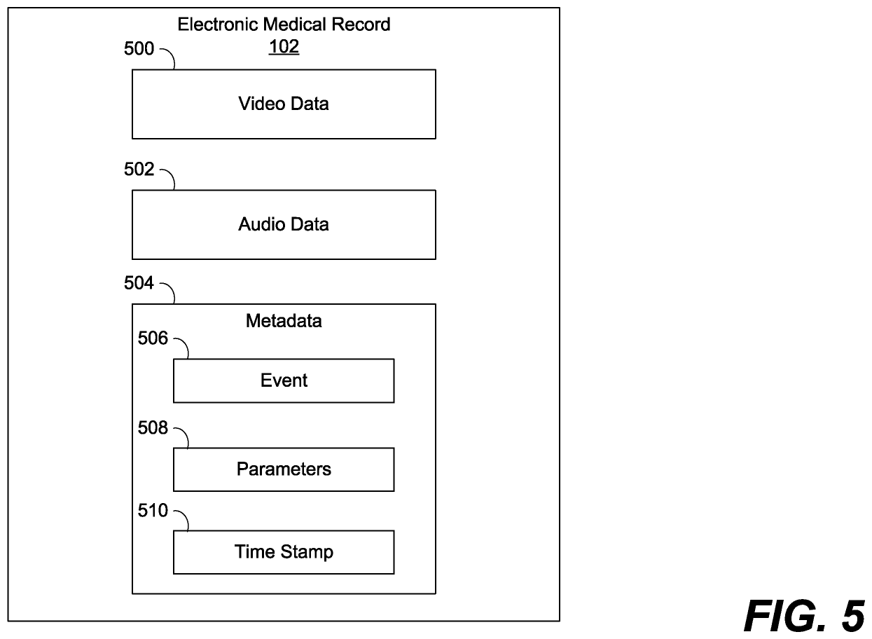

FIG. 5 schematically illustrates an example of the EMR 102. As shown in FIG. 5, the EMR 102 includes video data 500, audio data 502, and metadata 504. As described above, the video data 500 and the audio data 502 are captured, respectively, by the camera 202 and the microphone 214 during the data acquisition session performed by the AI interactive application 226. The metadata 504 includes identification of medically relevant events extracted from the video data 500 and the audio data 502. For example, the metadata 504 can include detection of an event 506, one or more parameters 508 measured during the event, and a time stamp 510 of when the event occurs in the video data 500 and the audio data 502.

As an illustrative example, the metadata 504 can include an event 506 of the subject S showing their wound in response to the interrogatories during the data acquisition session, one or more parameters 508 measured while the subject S is showing their wound such as wound size, whether the wound is wet or dry, whether dehiscence is present or not, and the like, and a time stamp 510 of when the subject S shows their wound in the video data 500 and the audio data 502.

As another example, the metadata 504 can include an event 506 of acquiring one or more vital signs measurements of the subject S, in which case the parameters 508 include the values of the one or more vital signs measurements, and a time stamp 510 of when the vital signs measurements are acquired from the subject S in the video data 500 and the audio data 502.

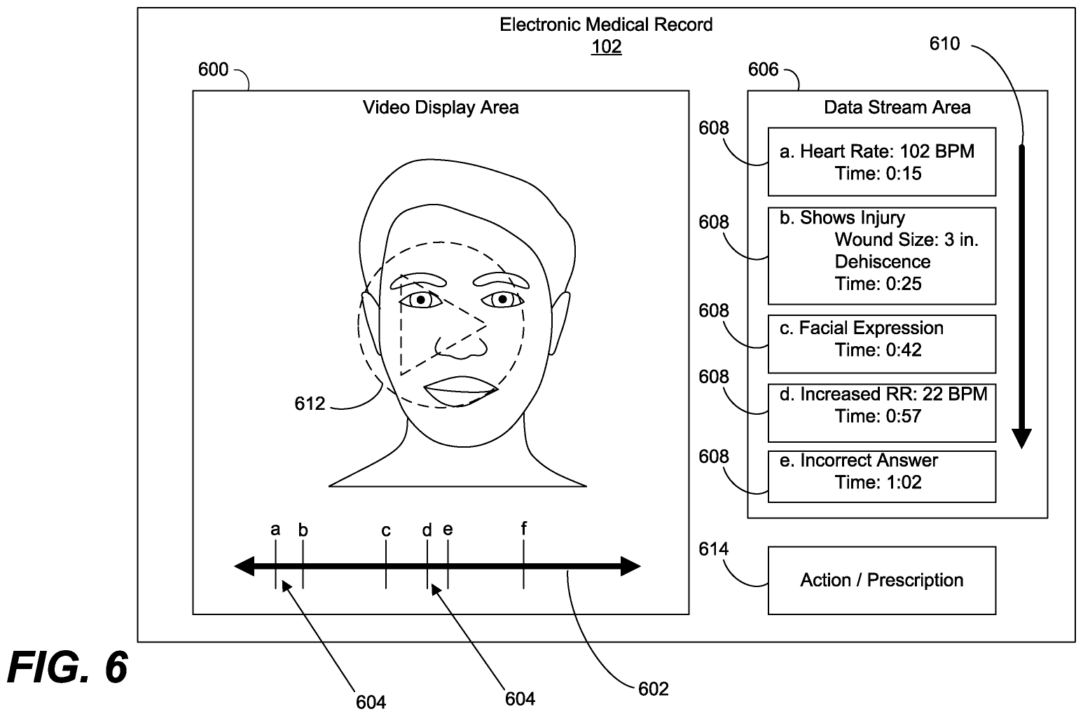

FIG. 6 illustrates an example of the EMR 102 that is generated by the system 10. As shown in FIG. 6, the EMR 102 includes a video display area 600 that displays the video data 500 captured by the camera 202. The video display area 600 includes a timeline 602 that ranges from the left side at the beginning of the video data 500 to the right side at the end of the video data 500. The timeline 602 within the video display area 600 includes medically relevant events 604 that correspond to medically relevant events 608 displayed within a data stream area 606 in the EMR 102. The data stream area 606 displays the medically relevant events 608 in chronological order. The data stream area 606 includes a scroll bar 610 that allows a user to scroll through the medically relevant events 608 displayed within the data stream area 606. The medically relevant events 608 can include a display of data that identifies the medically relevant event (e.g., acquired heart rate), one or more parameters or values associated with the medically relevant event (e.g., 102 beats per minute (BPM)), and a time stamp of the medically relevant event (e.g., 0:15) in the video data displayed in the video display area 600 of the EMR 102. Given the foregoing, the medically relevant events 608 are relevant to the clinical analysis of the subject S.

In the EMR 102, the user can select a medically relevant event 608 which causes the video display area 600 to automatically skip to the time stamp 510 of the medically relevant event 608. Alternatively, the user can select a medically relevant event 604 displayed on the timeline 602 within the video display area 600 which causes the video display area 600 to automatically skip to the time stamp 510 of the medically relevant event 604. Thereafter, the user can select a play icon 612 to play the video data 500 and the audio data 502 captured during the medically relevant event 608. Thus, the video data 500 and the audio data 502 captured by the data acquisition device 200 are part of the EMR 102, which provides a personalized human narrative to facilitate clinical assessment of the subject S.

A user such as a medical professional can view the video data 500 and the audio data 502 as well as the metadata 504 extracted from the video data 500 and the audio data 502 to provide a clinical assessment of the subject S, which resembles an in-person or virtual consultation even though the medical professional has never seen the subject S in-person, or was not present during the data acquisition session with the subject S. The video data 500 and the audio data 502 when viewed together with one or more vital signs and/or physical assessment attributes included in the metadata 504 provides a holistic view of the health status of the subject S that can result in more complete and accurate clinical assessments of the subject S.

The EMR 102 further displays an action area 614 that displays an action that was performed during the data acquisition session with the subject S, or a prescription that was recommended during the data acquisition session with the subject S. As an illustrative example, the action area 614 can display one or more follow-up tests, exams, referrals, or medical prescriptions that were recommended by the AI interactive application 226 based on the health information acquired from the data acquisition session with the subject S.

As another illustrative example, the action area 614 can display one or more actions that were performed by the AI interactive application 226 during the data acquisition session with the subject S such as switching on a dome light outside a patient room, issuing an alert to a nurse call system, increasing a rounding frequency, or increasing an early warning score (EWS) calculated for the subject S when the responses to the interrogatories or the parameters measured during the data acquisition session with the subject S indicate necessity for medical intervention.

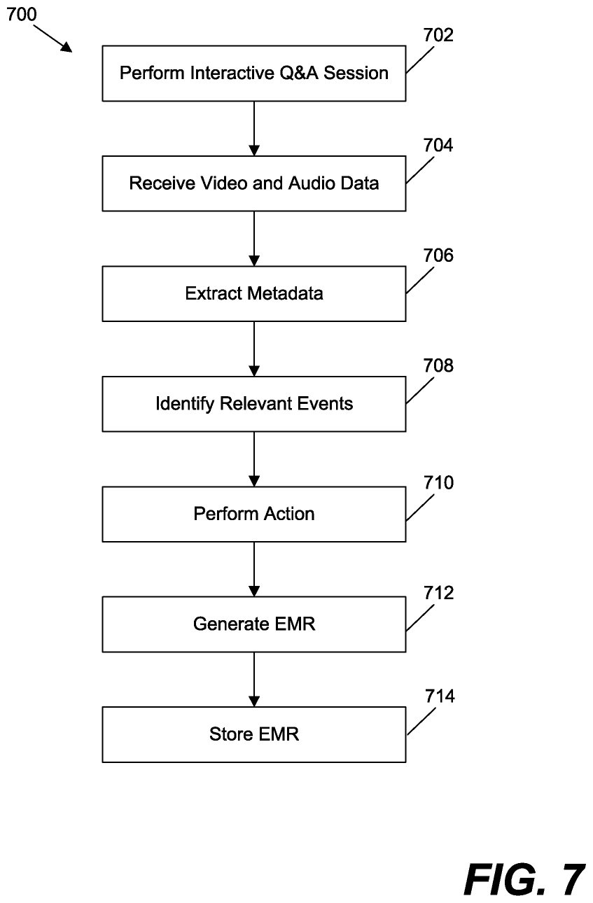

FIG. 7 schematically illustrates an example of a method 700 of providing virtual nursing through generating the EMR 102. The method 700 can be performed by the system 10. In some examples, the method 700 includes an operation 702 of performing the data acquisition session with the subject S. As described above, the AI interactive application 226 can display the avatar 402 on the display device 216 of the data acquisition device 200 to engage with the subject S. For example, the AI interactive application 226 adapts the interrogatories to have a fluid and free-flowing conversation with the subject S by asking a series of interrogatories that are adaptable based on the responses given by the subject S to prior interrogatories.

The method 700 includes an operation 704 of recording and/or receiving the video data 500 and the audio data 502 of the subject S. In some examples, the video data 500 and the audio data 502 are recorded and/or received during the data acquisition session performed by the AI interactive application 226. Operation 704 can include providing the data acquisition device 200 for operation by the subject S to record the video data 500 and the audio data 502.

The method 700 includes an operation 706 of extracting the metadata 504 from the video data 500 and the audio data 502. The metadata 504 can include an event 506, one or more parameters 508 measured during the event, and a time stamp 510 of when the event occurs in the video data 500 and the audio data 502. The metadata 504 is extracted by the video data extractor application 228. In examples where the video data extractor application 228 is installed on the data acquisition device 200, the metadata 504 is extracted locally on the data acquisition device. Alternatively, in examples where the video data extractor application 228 is installed on the analytics system 300, the analytics system 300 receives the video data 500 and the audio data 502 from the data acquisition device 200 via the communications network 20, and the metadata 504 is extracted by the analytics system 300 externally from the data acquisition device 200.

In some examples, the metadata 504 is extracted in real-time while the video data 500 and the audio data 502 is being recorded. In such examples, the interrogatories generated in the data acquisition session can be based on the metadata 504. In other examples, the metadata 504 is extracted after recording the video data 500 and the audio data 502 is completed.

The events 506 included in the metadata 504 can include measurements of one or more vital signs based on the video data 500 and the audio data 502 such as heart rate, respiration rate, blood pressure, blood oxygen saturation (SpO2), heart rate variability, body temperature, and the like. As an illustrative example, heart rate can be measured from the video data 500 such as by measuring subtle color changes or motions of the face of the subject S due to perfusion of blood by the heart, which are invisible to human eyes but can be captured by the camera 202 of the data acquisition device 200. As another example, blood pressure can be measured from the video data 500 by calculating a pulse propagation time difference or instantaneous phase difference between two pulse waves obtained from different parts of the subject S's body in the video data 500 captured by the camera 202. As a further example, SpO2 can be measured by extracting an imaging photoplethysmography (iPPG) signal from a region of interest on the skin such as the forehead of the subject S and decomposing the iPPG into red and green channels to obtain optical properties from these wavelengths associated with SpO2 measurements. As another example, the body temperature of the subject S can be measured using the thermal imaging modality 212 of the camera 202 of the data acquisition device 200.

Similarly, respiration rate can be measured from the video data 500 by detecting subtle motions around the chest area of the subject S due to expansion and contraction of the lungs from breathing. In further examples, the respiration rate can be measured from the audio data 502 which can by analyzed to detect breathing by the subject S. In further examples, the heart rate and respiration rate of the subject S can be measured based on reflected millimeter wave signals of the subject S detected by the millimeter wave antenna 204.

As a further example, events 506 included in the metadata 504 can include a cough by the subject S. In such examples, the cough can be detected from the audio data 502, which can be analyzed to determine one or more health conditions of the subject S such as whether the subject S is exhibiting mucus congestion, wheezing, asthma, or other lung conditions.

As a further example, events 506 included in the metadata 504 can include an erroneous response to an interrogatory during the data acquisition session performed by the AI interactive application 226. For example, the data acquisition session can include a series of requests for the subject S to move their eyes, speak, and move their body as part of a Glasgow Coma Scale (GCS) assessment. An event 506 can include when the subject S is unable to perform a requested eye or body movement, or when the subject S provides an incorrect verbal response to an interrogatory related to the Subject S's orientation to time, person, and place, or when the subject S is unable to converse normally with the avatar 402.

As a further example, events 506 included in the metadata 504 can include when the subject S shows an anatomical portion of their body such as a wound, rash, sore, or swelling. As a further example, events 506 included in the metadata 504 can include when the subject S exhibits a facial expression such as a grimace due to pain. As a further example, events 506 included in the metadata 504 can include posture or gait indicative of a medical condition.

The method 700 includes an operation 708 of identifying medically relevant events from the metadata 504 extracted in operation 706. As an example, operation 706 can include extracting a plurality of events some of which may be medically relevant, while others are not. In such cases, operation 708 can include filtering the plurality of events to identify only medically relevant events. Given the foregoing, the medically relevant events include events that are identified based on the metadata 504 as being relevant to a clinical analysis of the subject S. Illustrative examples of the medically relevant events are described above.

In some further examples, operation 708 includes editing the video data 500 and the audio data 502 to have a predetermined duration. As an illustrative example, operation 708 can include editing the video data 500 and the audio data 502 to have a duration of about 30 seconds. In such examples, operation 708 includes editing the video data 500 and the audio data 502 to provide a highlight reel of medically relevant events for playback in the video display area 600 of the EMR 102 (see FIG. 6). This allows the EMR 102 to present the health information of the subject S more efficiently and decreases memory storage requirements of the EMR system 100, while also providing a personalized human narrative to facilitate clinical assessment of the subject S. Given the foregoing, the EMR 102 improves the functioning of the EMR system 100.

The method 700 can include an operation 710 of performing an action based on the medically relevant events identified in operation 708. For example, when the subject S is a patient admitted to a medical facility such as a hospital or a long-term care facility, operation 710 can include switching on a dome light outside the subject S's room, triggering an audio and/or visible alarm on a medical device in proximity to the subject S, or issuing an alert to a nurse call system when a medically relevant event indicates that a medical intervention is necessary.

For example, when physical assessment attributes included in the metadata 504 indicate that a wound is leaking fluid, operation 710 can include issuing an alert to a nurse call system that the wound needs to be cleaned and rebandaged. As another example, when physical assessment attributes included in the metadata 504 indicate that an intravenous (IV) catheter has been pulled out (whether intentionally or by accident), operation 710 can include issuing an alert to a nurse call system that the IV needs to be re-inserted.

As further example, operation 710 can include increasing a nurse rounding frequency in the medical facility when a medically relevant event indicates a deterioration of the subject S's condition such as a lower early warning score (EWS) based on the one or more vitals and physical assessment attributes extracted from the video data 500 and the audio data 502. As another example, operation 710 can include issuing a prescription for pain relief medication (e.g., acetaminophen, ibuprofen, and the like) when physical assessment attributes in the metadata 504 (e.g., audible groaning, grimace facial expression) indicate the subject S is in pain.

As another example, operation 710 can include controlling a medical device in proximity to the subject S such as to adjust one or more settings based on the medically relevant invent. For example, operation 710 can include adjusting a dosage delivered by an infusion pump based on whether the vital signs and physical assessment attributes in the metadata 504 indicate that the subject S is responding to a prescribed treatment or not. As another example, operation 710 can include adjusting a frequency of vital sign acquisition by one or more sensors monitoring the subject S when the vital signs and physical assessment attributes in the metadata 504 indicate that the condition of the subject S is deteriorating. As a further example, operation 710 can include controlling a smart hospital bed to adjust a bed angle and/or facilitate patient turning when the physical assessment attributes in the metadata 504 indicate the subject S is likely to have bedsores and pressure injuries based on their posture.

As another example, such as when the subject S is located in their home, operation 710 can include providing a recommendation for one or more follow up medical office visits, exams, tests, or referrals, or issuing an alert to emergency medical services (EMS), ambulance services, or paramedic services, to provide urgent treatment and stabilization.

The method 700 includes an operation 712 of generating the EMR 102. The EMR 102 is generated by the EMR generator application 230. In examples where the EMR generator application 230 is installed on the data acquisition device 200, the EMR 102 is generated locally on the data acquisition device 200. Alternatively, in examples where the EMR generator application 230 is installed on the analytics system 300, the analytics system 300 receives the video data 500, the audio data 502, and the metadata 504 from the data acquisition device 200 via the communications network 20, and the EMR 102 is generated by the analytics system 300.

Operation 712 includes generating the EMR 102 to include the video display area 600 for providing playback of the video data 500 and the audio data 502 (see FIG. 6). In some examples, the video data 500 and the audio data 502 included in the video display area 600 have a duration of about 30 seconds. In other examples, the video data 500 and the audio data 502 included in the video display area 600 can have a duration longer than 30 seconds.

Operation 712 further includes generating the EMR 102 to include the data stream area 606 that displays the medically relevant events identified in operation 708. As described above, the medically relevant events 608 are selectable in the data stream area 606 which causes the video display area 600 to automatically skip to the portion of the video data 500 and the audio data 502 where a medically relevant event occurs based on the time stamp 510 in the metadata 504. Additionally, operation 712 can further include generating the EMR 102 to include the action area 614 that displays the action performed in operation 710.

The method 700 includes an operation 714 of storing the EMR 102. For example, operation 714 can include storing the EMR 102 in the EMR system 100. In such examples, the EMR 102 can be made accessible to authorized medical professionals across different medical facilities and settings via the communications network 20. Additionally, by storing the EMR 102 in the EMR system 100, the video data 500 and the audio data 502 in the EMR 102 can be run through future algorithms which may not exist at the time the video data 500 and the audio data 502 are originally recorded. Additionally, or alternatively to storing the EMR 102 in the EMR system 100, operation 714 can include storing the EMR 102 locally on the data acquisition device 200 allowing the subject S to have local access to the EMR 102.

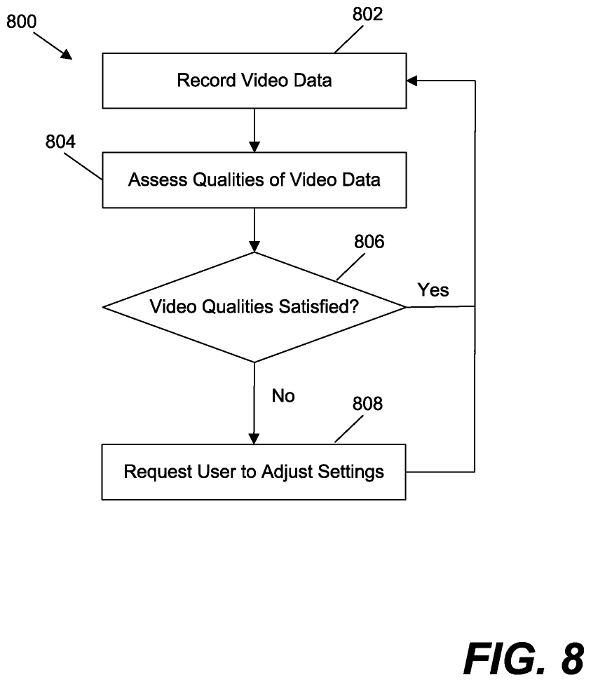

FIG. 8 schematically illustrates an example of a method 800 of generating the video data 500 that can be performed by the video quality evaluator application 232. The method 800 can be performed simultaneously with the method 700 of FIG. 7 or at least during certain operations of the method 700. For example, the method 800 can be performed during the data acquisition session with the subject S (operation 702). As will be discussed in more detail, the method 800 includes assessing the quality of the video data 500 in real-time to ensure that the video data 500 meets standards for optimal extraction of the metadata 504.

In examples where the video quality evaluator application 232 is installed on the data acquisition device 200, the method 800 can be performed locally on the data acquisition device 200. Alternatively, in examples where the video quality evaluator application 232 is installed on the analytics system 300, the method 800 can be performed by the analytics system 300.

The method 800 includes an operation 802 of recording the video data 500. As described above, the video data 500 can be recorded by the camera 202 of the data acquisition device 200. It is contemplated that operation 802 can include recording the video data 500 under any modality of the camera 202. For example, operation 802 can include recording the video data 500 under the RGB imaging modality 206, the infrared imaging modality 208, the depth imaging modality 210, or the thermal imaging modality 212. In some instances, operation 802 can include recording the video data 500 under two or more imaging modalities simultaneously. Operation 802 can be performed during the data acquisition session with the subject S.

The method 800 includes an operation 804 of assessing one or more qualities of the video data 500 in real-time. Operation 804 can include assessing a lighting intensity of the video data 500, a lighting variability (e.g., light reflections and/or light scatter) in the video data 500, identifying whether motion by the subject S or by objects around the subject S is present in the video data 500, identifying whether objects are obfuscating relevant anatomical portions of the subject S, and/or comparing the video data 500 to one or more standards such as Digital Imaging and Communications in Medicine (DICOM) and other types of standards.

The method 800 includes an operation 806 of determining whether the one or more qualities of the video data 500 (e.g., lighting intensity, lighting variability, motion, object obfuscation, and the like) satisfy one or more thresholds, or one or more standards (e.g., DICOM). For example, operation 806 can include determining whether the lighting intensity of the video data 500 satisfies a minimal threshold for lighting intensity. For example, the minimal threshold can be based on whether the video data 500 includes sufficient illumination for the video data extractor application 228 to extract the metadata 504 from the video data 500.

Operation 806 can also include determining whether the lighting variability in the video data 500 is below a maximum threshold allowed for the video data 500. For example, the maximum threshold for lighting variability can be based on whether the lighting variability interferes with the ability of the video data extractor application 228 to extract the metadata 504.

Operation 806 can also include determining whether motion by the subject S or by other objects around the subject S is below a maximum threshold allowed for the video data 500. For example, the maximum threshold for motion can be based on whether the motion interferes with the ability of the video data extractor application 228 to extract the metadata 504.

Operation 806 can also include determining whether objects are obfuscating a region of interest for the video data extractor application 228 to extract the metadata 504 such as objects obfuscating the face of the subject S, or a wound on the body of the subject S. Operation 806 can also include determining whether the video data 500 satisfies one or more standards such as Digital Imaging and Communications in Medicine (DICOM) and similar types of standards.

When the one or more qualities of the video data 500 satisfy one or more thresholds or one or more standards (i.e., “Yes” in operation 806), the method 800 returns to operation 802 such that the method 800 continues recording the video data 500.

Otherwise, when the one or more qualities of the video data 500 do not satisfy one or more thresholds or one or more standards (i.e., “No” in operation 806), the method 800 proceeds to operation 808 of instructing the subject S to adjust one or more settings to improve the one or more qualities of the video data 500. For example, when the light intensity of the video data 500 does not satisfy a minimal threshold, operation 808 can include instructing the subject S to turn on ambient lighting around them. As another example, when the motion of the subject S exceeds a maximum allowed threshold, operation 808 can include instructing the subject S to remain still. After instructing the subject S to adjust one or more settings in operation 808, the method 800 then returns to operation 802 such that the method 800 continues recording the video data 500.

As shown in FIG. 8, operations 802-808 are repeated such that the quality of the video data 500 is continuously assessed. As described above, in some examples, the quality of the video data 500 is continuously assessed during the data acquisition session. Also, while the method 800 is described with reference to the video data 500, it is contemplated that similar methods can be performed to ensure that the audio data 502 satisfies one or more quality metrics.

FIG. 9 schematically illustrates an example of a method 900 of performing a clinical assessment of the subject S. The method 900 can be performed by the system 10. The method 900 includes an operation 902 of initiating a data acquisition session with the subject S. As described above, initiating the data acquisition session can include presenting a simple question such as “How are you feeling?” or a similar type of introductory conversation starter.

The method 900 includes an operation 904 of analyzing the video data 500 and the audio data 502 captured by the data acquisition device 200. Operation 904 can include analyzing the audio data 502 when the subject S responds with a verbal reply (e.g., uttering “Not great”, “I have pain in my back”, “I'm feeling nauseous”, and the like), and/or can include analyzing the video data 500 when the subject S responds with a non-verbal reply (e.g., gesturing thumbs down, making facial expression, showing a wound, rash, sore, swelling, and the like). In further examples, operation 904 can include measuring one or more vital signs based on the video data 500 and the audio data 502 such as heart rate, respiration rate, blood pressure, blood oxygen saturation (SpO2), heart rate variability, body temperature, and the like.

The method 900 includes an operation 906 of determining one or more symptoms from the analysis of the video data 500 and the audio data 502. For example, operation 906 can include determining that the subject S is experiencing pain by analyzing the audio data 502 such as when the subject S utters a verbal response that they are experiencing pain, and/or when the subject S utters a sound such as groan indicative of pain. Operation 906 can also include determining that the subject S is experiencing pain by analyzing the video data 500 such as when the subject S makes a facial expression such as a grimace that is indicative of pain.

As another illustrative example, operation 906 can include determining that the subject S is experiencing an adverse medical condition by analyzing the video data 500 and the audio data 502 to determine that the subject S has an elevated heart rate or respiration rate.

The method 900 includes an operation 908 of generating one or more interrogatories based on the one or more symptoms determined in operation 906. For example, when operation 906 determines that the subject S is experiencing pain, operation 908 can include generating an interrogatory such as “Show me where it hurts.” As another example, when operation 906 determines that the subject S has an elevated heart rate or respiration rate, operation 908 can include generating an interrogatory such as “Take a deep breath and let's try to calm down. Please explain what happened.” As another example, when operation 906 determines that the subject S has a fever based on their body temperature, operation 908 can include generating an interrogatory such as “You have a fever. When did you first start experiencing symptoms?” Thus, the one or more vital signs and physical assessment attributes determined from the video data 500 and the audio data 502 can be used to generate the interrogatories, and thereby guide the data acquisition session with the subject S to acquire the most relevant health information.

The method 900 includes an operation 910 of outputting the one or more interrogatories generated in operation 908. For example, the interrogatories can be outputted via the avatar 402 displayed on the data acquisition device 200.

The method 900 includes an operation 912 of recording the video data 500 and the audio data 502 when the subject S responds to the interrogatories outputted in operation 910. Operation 912 can include recording the video data 500 and the audio data 502 of medically relevant events which include health information associated with the subject S. For example, operation 912 can include recording the video data 500 when the subject S shows an injury or wound in response to an interrogatory, and can also include the audio data 502 when the subject S describes their injury or wound in response to an interrogatory. Additionally, operation 912 can include extracting the metadata 504 from the video data 500 and the audio data 502, which can include events 506 including health information such as one or more vital signs (e.g., heart rate, respiration rate, blood pressure, blood oxygen saturation (SpO2), heart rate variability, body temperature, and the like), and/or physical assessment attributes (e.g., wound size, whether the wound is wet or dry, whether dehiscence is present or not, and the like).

The method 900 includes an operation 914 of determining whether a sufficient amount of health information is acquired from the subject S based on the recorded responses to the interrogatories in operation 912. For example, operation 914 can include determining whether the health information recorded in operation 912 meets a minimal threshold for generating the EMR 102. As another example, operation 914 can include determining whether the health information recorded in operation 912 meets a minimal threshold for generating a recommendation for the subject S such as to prescribe one or more follow up exams, tests, or referrals, or to perform an action related to the healthcare of the Subject S such as switching on or off a dome light outside a patient room, issuing or rescinding an alert to a nurse call system, adjusting a rounding frequency, or adjusting an early warning score (EWS) of the subject S.

When the health information recorded in operation 912 does not meet a threshold (i.e., “No” in operation 914), the method 900 can include repeating the operations 904-914 to acquire additional health information of the subject S. When the health information recorded in operation 912 satisfies the threshold (i.e., “Yes” in operation 914), the method 900 can proceed to operation 916 of terminating the data acquisition session with the subject S.

After completion of the data acquisition session, the EMR 102 is generated based on the health information acquired from performance of the method 900. Also, one or more recommendations for the subject S such as one or more follow up exams, tests, or referrals can be generated, or one or more actions related to the healthcare of the Subject S can be performed based on the health information acquired from performance of the method 900. The method 900 allows clinical assessment of the subject S when there are limited medical resources such as when the subject S is in their home, or when the subject S is admitted to a healthcare facility that is exhibiting a staff shortage. Also, the method 900 allows remote monitoring of the subject S via virtual nursing, such that it can be utilized in a home hospital program that provides in-home care for persons who need the services and care that hospitalization provides, but who are stable enough to be monitored in the comfort of their own homes.

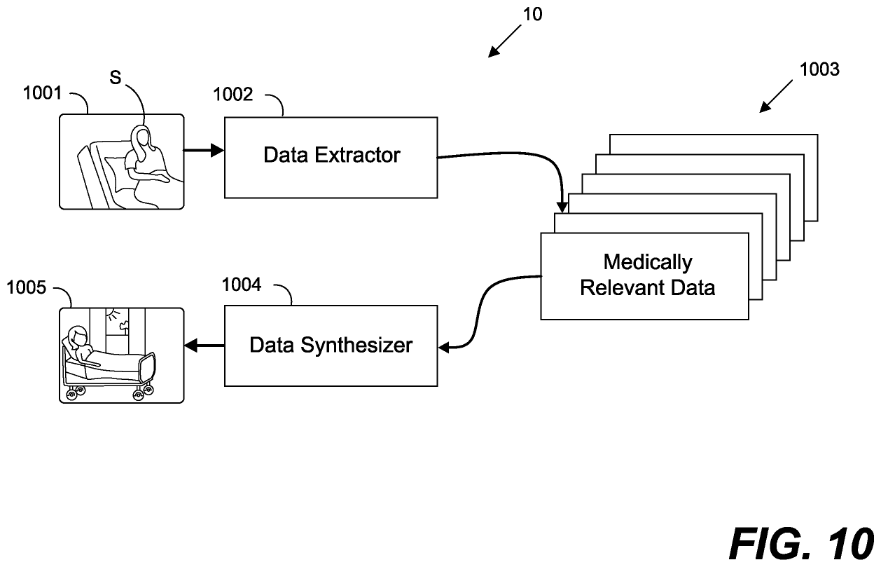

FIG. 10 schematically illustrates another example of the system 10 that can further enhance the EMR 102 stored in the EMR system 100 (see FIG. 1). Aspects of the system 10 can be implemented on the data acquisition device 200 and/or on the analytics system 300.

As will be described in more detail, the system 10 reduces digital storage space requirements for storing the video data and the audio data captured by the data acquisition device 200 in the EMR 102 of the subject S. Additionally, the system 10 removes data that could be used to identify the subject S to improve privacy and confidentiality of the video data and the audio captured by the data acquisition device 200. Further, the system 10 can reduce or eliminate subconscious biases of medical professionals who review the EMR 102 of the subject S by excluding data that identifies the race, ethnicity, or other medically unrelated characteristics of the subject S. Also, the system 10 standardizes the video data and the audio data captured by the data acquisition device 200 across a plurality of EMRs 102 stored in the EMR system 100.

The system 10 includes a data extractor 1002 that receives raw data 1001 of the subject S captured by the data acquisition device 200. The raw data 1001 can include video data captured by the camera 202. The raw data 1001 can include an RGB video of the subject S captured by the RGB imaging modality 206. Additionally, or alternatively, the raw data 1001 can include video data captured by other imaging modalities of the camera 202. The raw data 1001 can also include audio data captured by the microphone 214 of the data acquisition device 200.

The data extractor 1002 extracts medically relevant data 1003 from the raw data 1001 while excluding other data that is not medically relevant. The data extractor 1002 is implemented on the data acquisition device 200 such that the data extraction is done locally to protect the identity of the subject S. The medically relevant data 1003 is then communicated to the EMR system 100 or the analytics system 300 over the communications network 20 (FIG. 1). The data extractor 1002 can include aspects of the video data extractor application 228 described above.

The data extractor 1002 is configurable to one or more health conditions of the subject S that are being monitored by the system 10 such that the medically relevant data 1003 extracted by the data extractor 1002 varies based on the one or more health conditions of the subject S. As an illustrative example, the system 10 can automatically identify the one or more health conditions of the subject S through analysis of the video data and the audio data captured by the data acquisition device 200. The system 10 can further identify the one or more health conditions of the subject S by accessing via the communications network 20 the EMR 102 of the subject S where the one or more health conditions of the subject S are stored. Alternatively, the one or more health conditions of the subject S can be entered manually on the data acquisition device 200 by the subject S or by a caregiver providing healthcare services to the subject S.

The one or more health conditions of the subject S can include one or more medical conditions, diagnoses, and/or comorbidities. The one or more health conditions of the subject S can include risks identified for the subject S such as whether the subject S is at risk for falling. The one or more health conditions of the subject S can also include a status and/or location of the subject S such as whether the subject S is located in a pre-operative holding area (i.e., before surgery) or in a post-surgical recovery room (i.e., after surgery). Thus, the data extractor 1002 is configurable to the one or more health conditions of the subject S where the medically relevant data 1003 extracted by the data extractor 1002 can vary based on relevancy to monitoring for a specific health condition. For example, the medically relevant data 103 extracted by the data extractor 1002 can vary for post-surgical physical therapy monitoring versus level of consciousness monitoring versus mobility monitoring, and other health conditions.

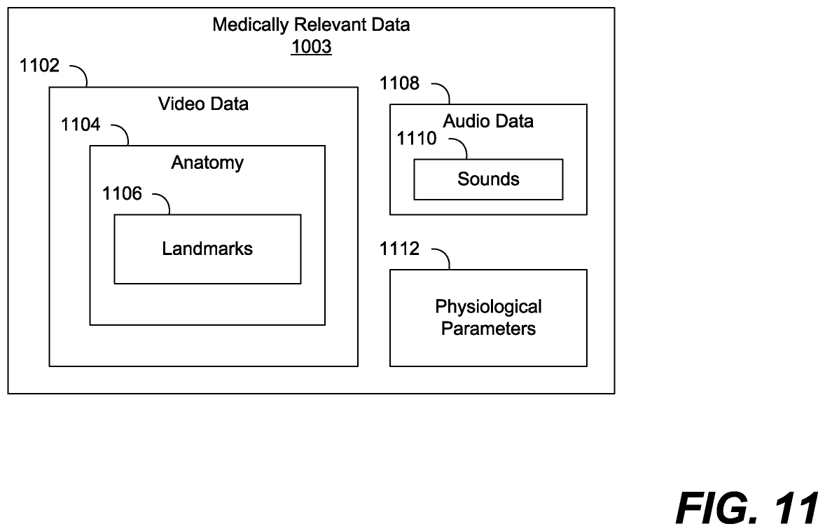

FIG. 11 schematically illustrates an example of the medically relevant data 1003 extracted by the data extractor 1002 based on the one or more health conditions of the subject S. In this example, the raw data 1001 includes video data 1102 and audio data 1108. The medically relevant data 1003 extracted from the raw data 1001 includes one or more landmarks 1106 within an anatomy 1104 captured in the video data 1102. The medically relevant data 1003 can also include one or more sounds 1110 captured in the audio data 1108 such as verbal responses to interrogatories, slurred speech, coughing, wheezing, sneezing, and other audible sounds from the subject S. Also, the medically relevant data 1003 can include one or more physiological parameters 1112 detected or calculated from the video data 1102 and/or the audio data 1108. The medically relevant data 1003 is provided by way of illustrative example such that additional types of medically relevant data, or fewer types of medically relevant data may be extracted.

As an illustrative example, the data extractor 1002 is configurable to extract medically relevant data 1003 for an eye response evaluation which is part of the Glasgow coma scale. The medically relevant data 1003 extracted from the raw data 1001 includes an anatomy 1104 of the subject S's face, and landmarks 1106 such as the left pupil and the right pupil.

The data extractor 1002 is also configurable to extract medically relevant data 1003 for a verbal response evaluation which is also part of the Glasgow coma scale. In such example, the medically relevant data 1003 extracted from the raw data 1001 can also include sounds 1110 extracted from the audio data 1108 such as verbal replies to interrogatories that evaluate how well certain brain abilities work, including thinking, memory, attention span and awareness of surroundings. The data extractor 1002 can extract medically relevant data from the audio data 1108 such as a timeliness of the verbal responses to the interrogatories and slurred speech.

As another example, when the one or more health conditions of the subject S include a falling risk, the data extractor 1002 is configurable to extract the medically relevant data 1003 for a mobility assessment. In such an example, the medically relevant data 1003 extracted from the raw data 1001 includes an anatomy 1104 of the subject S's entire body, and landmarks 1106 include the shoulders, knees, ankles, and other body parts. Also, the data extractor 1002 may also extract one or more physiological parameters 1112 from the raw data 1001 that may be relevant to the mobility assessment such as the subject S's body weight, height, and the like.

As another illustrative example, when the system 10 is monitoring for a stroke recovery, the data extractor 1002 extracts landmarks 1106 from an anatomy 1104 that includes the subject S's face for assessing whether a portion of the subject S's face is paralyzed.

As another illustrative example, when the system 10 is monitoring for a rash, the data extractor 1002 extracts data relevant for assessing the rash such as a location of the rash, a coloring of the rash, and other relevant characteristics. In some instances, the data extractor 1002 extracts an RGB video or a still image of the rash from the raw data 1001 while other body portions shown or depicted in the raw data 1001 are excluded.

As a further illustrative example, the data extractor 1002 extracts volumetric information of one or more body parts. For example, the data extractor 1002 can extract a volume measurement of a body part that is exhibiting swelling (e.g., left hand) and can also extract a volume measurement from another body part that is not exhibiting swelling (e.g., right hand) for a relative comparison. The volume measurements can be extracted from the depth data captured by the depth imaging modality 210 of the camera 202.

As another illustrative example, when monitoring for a mobility assessment, the data extractor 1002 can extract medically relevant data 103 related to mobility such as a distance walked by the subject S without assistance. The medically relevant data 103 can also include environmental factors such as whether a mobility aid such as a walker or cane was used by the subject S, or whether another person assisted the subject S when walking.

Referring back to FIG. 10, system 10 further includes a data synthesizer 1004 that synthesizes the medically relevant data 1003 to generate synthetic data 1005. Thereafter, the synthetic data 1005 is stored in the EMR 102 of the subject S. The data synthesizer 1004 can be implemented on the data acquisition device 200 such that the synthesis of the medically relevant data 1003 is performed locally on the data acquisition device 200. Alternatively, the data synthesizer 1004 can be implemented on the analytics system 300 such that the synthesis of the medically relevant data 1003 is performed externally from the data acquisition device 200. The data extractor 1002 can include aspects of the EMR generator application 230 described above.

The synthetic data 1005 de-identifies the subject S such as by excluding details that identify the subject S. For example, video data that shows the subject S's face and audio data that includes the subject S's voice are replaced with the synthetic data 1005. Further, by replacing the video data and the audio data with the synthetic data 1005, the race and ethnicity of the subject S are obscured such that unconscious biases of medical professionals who review the EMR 102 of the subject S are mitigated. The synthetic data 1005 also standardizes the video data and the audio data captured by the data acquisition device 200 when included in the EMRs of a plurality of subjects stored in the EMR system 100 because only medically relevant information distinguishes the synthetic data 1005 from subject to subject while other information is excluded.

Additionally, the synthetic data 1005 has a smaller data size than the raw data 1001 since it excludes the video data and the audio data recorded by the data acquisition device 200 that is not medically relevant to the one or more health condition of the subject S. Thus, the synthetic data 1005 reduces the digital storage space requirements for the EMR 102 on the EMR system 100 while allowing all medically relevant data to be retrievable for viewing.

Like the data extractor 1002, the data synthesizer 1004 is also configurable to the one or more health conditions of the subject S such that the synthetic data 1005 generated by the data synthesizer 1004 can vary based on the one or more health conditions of the subject S.

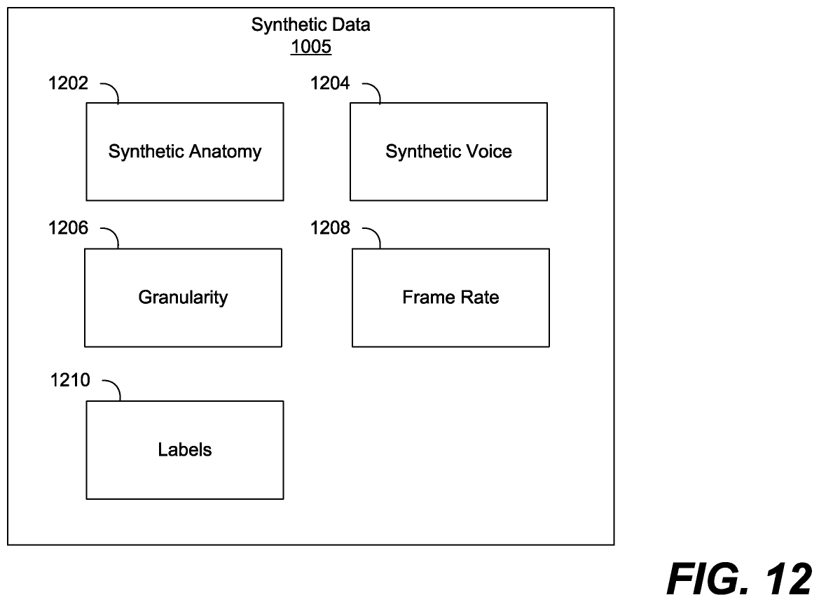

FIG. 12 schematically illustrates an example of the synthetic data 1005 generated by the data synthesizer 1004 based on the one or more health conditions of the subject S. In this example, the synthetic data 1005 can include a synthetic anatomy 1202 constructed based on the landmarks 1106 extracted by the data extractor 1002. The synthetic anatomy 1202 mimics the subject S's physical responses to the interrogatories in the data acquisition session.

The synthetic data 1005 can also include a synthetic voice 1204 constructed based on the sounds 1110 extracted from the audio data 1108. The synthetic voice 1204 mimics the subject S's verbal responses to the interrogatories in the data acquisition session.

The synthetic data 1005 can further include a granularity 1206 that can be based on a quantity of landmarks that are synthesized in the synthetic data 1005. For example, a larger quantity of landmarks means a higher granularity or level of detail, and a smaller quantity of landmarks means a lower granularity or level of detail. The granularity can depend on the health condition of the subject S that is monitored or assessed by the system 10 where certain health conditions may have higher granularity requirements, while other health conditions may have lower granularity requirements. By increasing the granularity, more details can be included in the EMR 102, and by lowering the granularity, the file size of the EMR 102 is reduced.

The synthetic data 1005 can further include a frame rate 1208 which can be based on frames per second (FPS). The frame rate 1208 is adjustable allowing for optimal evaluation of a health condition of the subject S. For example, the frame rate 1208 can be lowered to provide a slow motion replay of a health condition detected in the raw data 1001. Alternatively, the frame rate 1208 can be increased such as when the health condition detected in the raw data 1001 occurs over an elongated period of time. The frame rate 1208 can be adjusted to allow for optimal evaluation of the synthetic anatomy 1202 and/or the synthetic voice 1204.

The synthetic data 1005 can further include labels 1210 that identify one or more conditions detected in the raw data 1001 for synchronization with the synthetic data 1005. The labels 1210 can identify medically relevant events such as facial expressions, physical responses to the interrogatories, and verbal responses to the interrogatories. The labels 1210 can further identify physiological parameters 1112 detected from the video data 1102 and/or the audio data 1108 including vital signs, body weight, height, and the like.

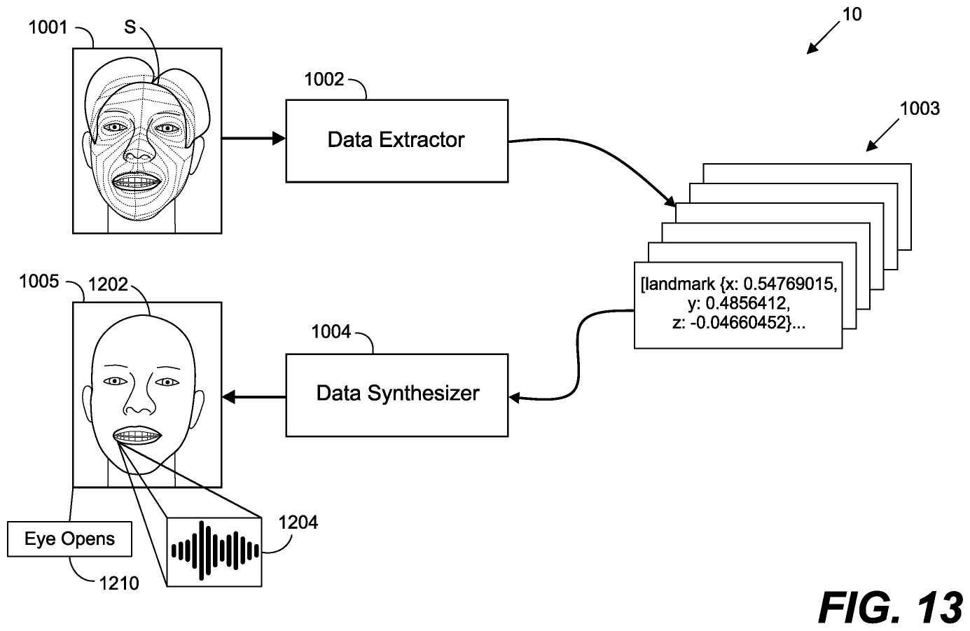

FIG. 13 schematically illustrates an example of the synthetic data 1005 generated by the data synthesizer 1004 that is optimally relevant for a level of consciousness assessment or a pain level assessment. In this illustrative example, the synthetic data 1005 includes a synthetic anatomy 1202 which is a face that includes landmarks such as a left pupil, a right pupil, a mouth, a nose, and ears. The synthetic data 1005 further includes a synthetic voice 1204 that is constructed from the sounds extracted from the audio data 1108. The synthetic voice 1204 can preserve details such as timeliness of a response to an interrogatory and slurred speech. All other information not relevant to evaluating level of consciousness or pain is excluded from the synthetic data 1005. The granularity 1206 can include a predetermined quantity of landmarks in the synthetic anatomy 1202, and the frame rate 1208 can be set for 30 frames per second (FPS) allowing for an objective evaluation of consciousness or pain experienced by the subject S.

The synthetic data 1005 can further include labels 1210 for one or more sequences of frames included in the synthetic anatomy 1202. For example, the labels 1210 can identify an interrogatory or detected response to an interrogatory relevant to the level of consciousness or pain of the subject S. The labels 1210 can also identify conditions detected in the raw data 1001 that are synchronized to the synthetic data 1005 such as a grimace.

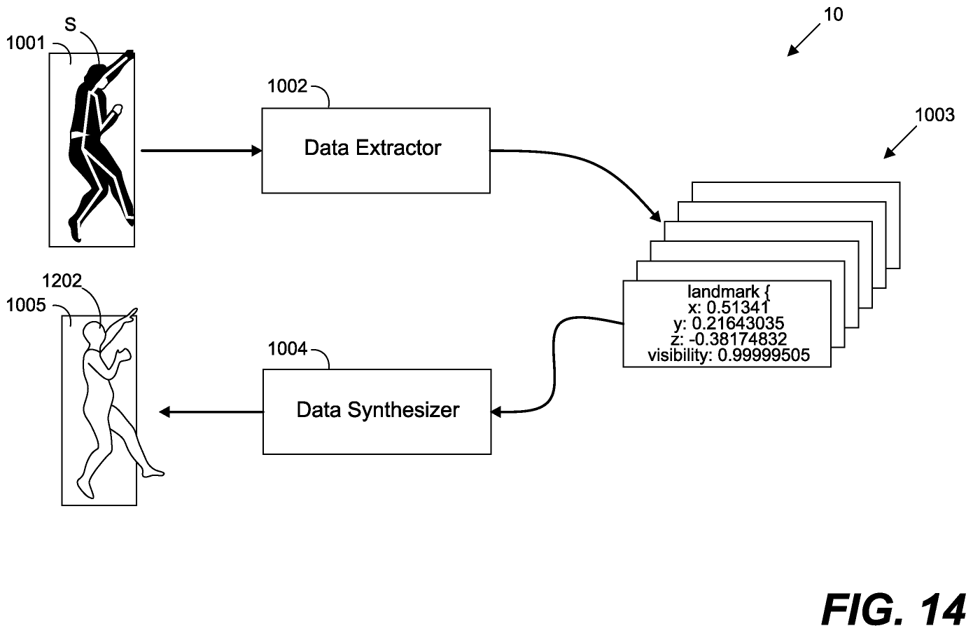

FIG. 14 schematically illustrates an example of the synthetic data 1005 generated by the system 10 when a health condition of the subject S indicate that the subject S is at risk for falling. In this illustrative example, the data synthesizer 1004 generates the synthetic data 1005 to be optimally relevant for a fall risk assessment and/or monitoring. In such example, the synthetic anatomy 1202 is the subject S's body while resting on a bed. The synthetic anatomy 1202 can include landmarks such as shoulders, knees, ankles, and other body parts. The granularity 1206 can include a quantity of landmarks in the synthetic anatomy 1202, and the frame rate 1208 can be set to 20 FPS allowing for optimal evaluation of the fall risk assessment and/or monitoring.

In further examples, when the system 10 is monitoring for a rash, the data synthesizer 1004 synthesizes data relevant for assessing the rash such as a location of the rash, a coloring of the rash, and other relevant characteristics. In some instances, the data synthesizer 1004 displays an RGB video or a still image of the rash overlayed on a synthetic anatomy 1202.

As a further illustrative example, when the system 10 is monitoring for swelling, the data synthesizer 1004 synthesizes volumetric information of one or more body parts. For example, the data synthesizer 1004 can generate a first synthetic anatomy based on a volume measurement of a body part extracted from the depth data captured by the depth imaging modality 210 of the camera 202, and can generate a second synthetic anatomy based on a volume measurement of another body part extracted from the depth data. The first and second synthetic anatomies can be displayed side-by-side to show relative swelling for one body part (e.g., left hand) compared to another body part (e.g., right hand). Advantageously, the synthetic anatomies provide an improved visualization that is more intuitive and easier to interpret than a measurement value or a score indicating a degree of swelling exhibited by the subject S.

As another illustrative example, when monitoring for a mobility assessment, the data synthesizer 1004 can synthesize the medically relevant data 103 to generate a synthetic anatomy 1202 showing a distance walked by the subject S. The data synthesizer 1004 can further generate avatars of environmental factors such as a mobility aid (e.g., walker or cane) used by the subject S when walking, or an avatar of another person who assisted the subject S to walk.

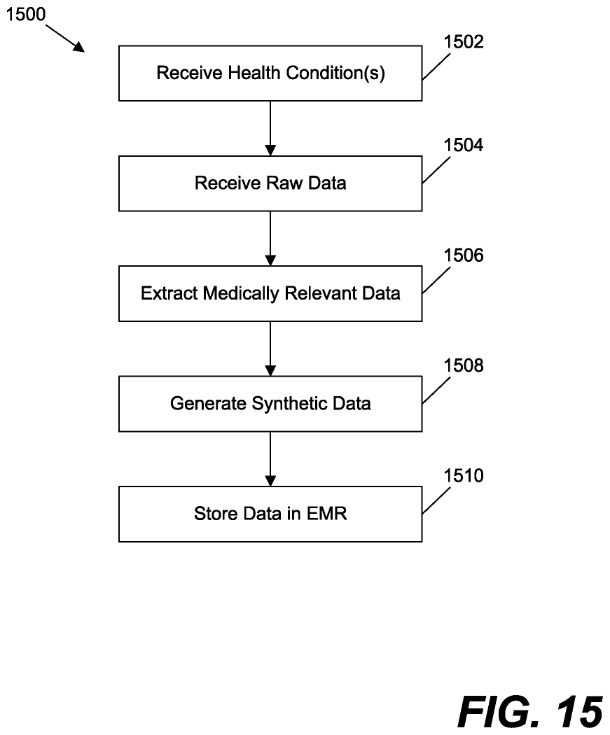

FIG. 15 schematically illustrates an example of a method 1500 of generating synthetic data for playback in the EMR 102 of the subject S. In some examples, the method 1500 is performed by the example of the system 10 as shown in FIGS. 10, 13, and 14.

The method 1500 includes an operation 1502 of receiving one or more health conditions of the subject S. The one or more health conditions can be received via an analysis of the video data 1102 and the audio data 1108 included in the raw data 1001 captured by the data acquisition device 200. Alternatively, the one or more health conditions can be received over the communications network 20 from the EMR 102 of the subject S. Alternatively, the one or more health conditions of the subject S can be received from a manual entry on the data acquisition device 200 by the subject S or by a caregiver providing healthcare services to the subject S.

The method 1500 includes an operation 1504 of receiving the raw data 1001. As described above, the raw data 1001 is captured by the data acquisition device 200. The raw data 1001 can include video data captured by one or more of the imaging modalities of the camera 202. The raw data 1001 can also include audio data captured by the microphone 214.

The method 1500 includes an operation 1506 of extracting the medically relevant data 1003 from the raw data 1001. Operation 1506 can be performed by the data extractor 1002 which extracts the medically relevant data 1003 based on the one or more health conditions of the subject S received in operation 1502. Operation 1506 can include extracting one or more landmarks 1106 from an anatomy 1104 based on the one or more health conditions. Operation 1506 can further include extracting one or more sounds 1110 from the audio data 1108 based on the one or more health conditions. Additionally, operation 1506 can include extracting based on the one or more health conditions one or more physiological parameters 1112 from the video data 1102 and/or the audio data 1108 captured by the data acquisition device 200. Operation 1506 can further include extracting additional data including metadata that is relevant to the one or more health conditions of the subject S received in operation 1502.

The method 1500 includes an operation 1508 of generating the synthetic data 1005 using the medically relevant data 1003 extracted from the raw data 1001 in operation 1506. Operation 1506 can be performed by the data synthesizer 1004 which generates the synthetic data 1005 based on the one or more health conditions of the subject S received in operation 1502.

Operation 1508 can include constructing a synthetic anatomy 1202 based on the landmarks 1106 extracted in operation 1506. The synthetic anatomy 1202 mimics the subject S's physical responses to the interrogatories in the data acquisition session.

Operation 1508 can also include constructing a synthetic voice 1204 based on the sounds 1110 extracted from the audio data 1108. The synthetic voice 1204 mimics the subject S's verbal responses to the interrogatories in the data acquisition session.

Additionally, operation 1508 can include adding one or more labels 1210 that are synchronized to the synthetic anatomy 1202 and the synthetic voice 1204. The one or more labels 1210 can include medically relevant events and physiological parameters 1112 detected from the video data 1102 and/or the audio data 1108 captured by the data acquisition device 200.

The method 1500 includes an operation 1510 of storing in the EMR 102 of the subject S the medically relevant data 1003 extracted in operation 1506 from the raw data 1001. The medically relevant data 1003 can include the one or more landmarks 1106 extracted from the anatomy 1104, the one or more sounds 1110 extracted from the audio data 1108, the one or more physiological parameters 1112 extracted from the video data 1102 and/or the audio data 1108, and other metadata that is synthesized to generate the synthetic data 1005. By storing the medically relevant data 1003 extracted from the raw data 1001, the method 1500 reduces digital storage space requirements for storing the EMR 102 on the EMR system 100.

In alternative examples, operation 1510 can include storing the synthetic data 1005 in the EMR 102 of the subject S. In such examples, the synthetic data 1005 is readily available for playback on demand by a user of the EMR system 100.

By replacing the video data and the audio data captured by the data acquisition device 200 with the synthetic data 1005 that can include the synthetic anatomy 1202 and the synthetic voice 1204, the race, ethnicity, and other non-medically relevant characteristics of the subject S are obscured such that unconscious biases of medical professionals who review the EMR 102 of the subject S are mitigated. Additionally, the synthetic data 1005 standardizes the video data and the audio data captured by the data acquisition device 200 because only the medically relevant data 1003 distinguishes the synthetic data 1005 from subject to subject.

By storing the medically relevant data 1003 in the EMR 102 of the subject S, the medically relevant data 1003 can be synthesized on demand such that the synthetic anatomy 1202 can be displayed in the video display area 600 (see FIG. 6). Additionally, one or more labels 1210 can be generated and applied to the synthetic anatomy 1202 and the synthetic voice 1204 to identify the medically relevant events 604, 608 in the video display area 600. This allows a user to select a medically relevant event 604, 608 which causes the video display area 600 to automatically skip to the time stamp 510 of the medically relevant event 604, 608. The user can then select a play icon 612 to play the synthetic anatomy 1202 and the synthetic voice 1204.

By providing playback of the synthetic anatomy 1202 and the synthetic voice 1204 in the EMR 102 of the subject S, the system 10 provides a personalized human narrative to facilitate clinical assessment of the subject S while at the same time excluding data that identifies the subject S to improve privacy and confidentiality of the video data and the audio captured by the data acquisition device 200 during the data acquisition session.

The synthetic data 1005 generated by the system 10 and the method 1500 is an improvement over text translation or scoring of the video data and the audio data captured by the data acquisition device 200 because the synthetic data 1005 preserves medically relevant details that would otherwise be lost by the text translation or scoring. Further, the synthetic data 1005 that includes the synthetic anatomy 1202 and/or the synthetic voice 1204 provides a personalized human narrative that is not possible via text translation or scoring. The personalized human narrative facilitates objective clinical assessment of the subject S by different clinicians.

The synthetic data 1005 generated by the system 10 and the method 1500 is an improvement over the raw data 1001 captured by the data acquisition device 200 because the synthetic data 1005 excludes data that could be used to identify the subject S thereby protecting the privacy and confidentiality of the subject S, while also mitigating subconscious biases by excluding data that identifies the race and ethnicity of the subject S. Additionally, by excluding data that is not medically relevant to the one or more health conditions of the subject S, the file size of the EMR 102 (in comparison to when the raw data 1001 is included in the EMR 102) can be reduced which improves data governance efficiency and data communications speed.

The synthetic data 1005 generated by the system 10 and the method 1500 is an improvement over the medically relevant data 1003 extracted from the raw data 1001 because by synthesizing the medically relevant data 1003, the synthetic data 1005 is easier to interpret by clinicians, and thereby facilitates objective clinical assessments by one or more clinicians.

The various embodiments described above are provided by way of illustration only and should not be construed to be limiting in any way. Various modifications can be made to the embodiments described above without departing from the true spirit and scope of the disclosure.

What is claimed is:

1. A system for generating an electronic medical record, the system comprising:
at least one processing device; and
at least one computer readable data storage device storing software instructions that, when executed by the at least one processing device, cause the system to:
receive video data and audio data of the subject;
extract metadata from the video data and the audio data, the metadata including an event, one or more parameters measured during the event, and a time stamp of when the event occurs in the video data and the audio data; and
generate an electronic medical record that includes a video display area for providing playback of the video data and the audio data, and a data stream area that displays one or more events in the video data and the audio data, at least one event of the one or more events when selected causes the video display area to skip to a portion of the video data and the audio data where the at least one event occurs based on the time stamp in the metadata associated with the at least one event, and wherein the at least one event is identified based on the metadata as relevant to the clinical analysis of the subject.

2. The system of claim 1, wherein the instructions, when executed by the at least one processing device, further cause the system to:
conduct a data acquisition session with the subject, the data acquisition session including interrogatories to guide acquisition of the video data and the audio data.

3. The system of claim 2, wherein the instructions, when executed by the at least one processing device, further cause the system to:
present an avatar controlled by artificial intelligence for conducting the data acquisition session with the subject.

4. The system of claim 2, wherein the instructions, when executed by the at least one processing device, further cause the system to:
adapt the interrogatories based on verbal and nonverbal responses given by the subject such that the data acquisition session is non-structured.

5. The system of claim 1, wherein the instructions, when executed by the at least one processing device, further cause the system to:
determine one or more vital signs and physical assessment attributes of the subject based on the video data and the audio data of the subject.

6. The system of claim 1, wherein the instructions, when executed by the at least one processing device, further cause the system to:
provide a recommendation for one or more follow-up tests, exams, referrals, or medical prescriptions based on the metadata extracted from the video data and the audio data.

7. The system of claim 1, wherein the video data includes one or more imaging modalities of a plurality of imaging modalities including a video imaging modality, an infrared imaging modality, a depth imaging modality, and a thermal imaging modality.

8. The system of claim 1, wherein the instructions, when executed by the at least one processing device, further cause the system to:
edit the video data and the audio data to have a predetermined duration.

9. A device for providing a clinical analysis of a subject, the device comprising:
at least one processing device; and
at least one computer readable data storage device storing software instructions that, when executed by the at least one processing device, cause the at least one processing device to:
conduct a data acquisition session including presenting interrogatories to guide acquisition of video data and audio data from the subject;
capture the video data and the audio data from the subject;
determine one or more vital signs and physical assessment attributes of the subject based on the video data and the audio data captured from the subject; and
extract metadata from the video data and the audio data, the metadata including an event, one or more parameters measured during the event, and a time stamp of when the event occurs in the video data and the audio data.

10. The device of claim 9, wherein the instructions, when executed by the at least one processing device, further cause the at least one processing device to:
adapt the interrogatories based on verbal and nonverbal responses given by the subject such that the data acquisition session is non-structured.

11. The device of claim 9, wherein the instructions, when executed by the at least one processing device, further cause the at least one processing device to:
present an avatar controlled by artificial intelligence to conduct the data acquisition session with the subject.

12. The device of claim 9, wherein the instructions, when executed by the at least one processing device, further cause the at least one processing device to:
provide a recommendation for one or more follow-up tests, exams, referrals, or medical prescriptions based on the metadata extracted from the video data and the audio data.

13. The device of claim 9, wherein the instructions, when executed by the at least one processing device, further cause the at least one processing device to:
generate an electronic medical record that includes a video display area for providing playback of the video data and the audio data, and a data stream area that displays one or more events in the video data and the audio data, at least one event of the one or more events when selected causes the video display area to skip to a portion of the video data and the audio data where the at least one event occurs based on the time stamp in the metadata.

14. A method of generating an electronic medical record, the method comprising:
receiving a health condition of a subject;
receiving raw data including at least video data captured of the subject;
extracting landmarks from an anatomy shown in the video data based on the health condition of the subject;
generating synthetic data including at least a synthetic anatomy constructed based on the landmarks extracted from the anatomy shown in the video data; and
storing the landmarks in the electronic medical record of the subject.

15. The method of claim 14, further comprising:
conducting a data acquisition session with the subject, the data acquisition session including interrogatories to guide acquisition of the video data; and
wherein the synthetic anatomy mimics physical responses by the subject to the interrogatories included in the data acquisition session.

16. The method of claim 14, further comprising:
extracting sounds from audio data of the subject included in the raw data, the sounds being extracted based on the health condition of the subject; and
generating a synthetic voice based on the sounds extracted from the audio data.

17. The method of claim 14, wherein the synthetic data has a granularity based on the health condition of the subject.

18. The method of claim 14, wherein the synthetic data has a frame rate based on the health condition of the subject.

19. The method of claim 14, further comprising:
generating labels for the synthetic data, the labels identifying medically relevant events based on the health condition of the subject.

20. The method of claim 14, wherein the synthetic data includes physiological parameters extracted from the raw data based on the health condition of the subject.
